(19) United States
(12) Patent Application Publication
(10) Pub. No.: US 2012/0253350 A1
(43) Pub. Date: Oct. 4, 2012

(54) BONE GRAFT SHAPER

(75) Inventors: SARAH M. ANTHONY, WARSAW, IN (US); JOSEPH P. IANNOTTI, WARSAW, IN (US); KYLE E. LAPPIN, WARSAW, IN (US); MATTHEW T. STONE, WARSAW, IN (US); JOANNA L. SURMA, WARSAW, IN (US); DAVID M. WARLOP, WARSAW, IN (US)

(73) Assignee: DEPUY PRODUCTS, INC., WARSAW, IN (US)

(21) Appl. No.: 13/077,196

(22) Filed: Mar. 31, 2011

(51) Int. Cl. A61B 17/56

(52) U.S. Cl. 606/87

(57) ABSTRACT

A bone graft shaper for shaping bone graft. The bone graft shaper includes a first holder having at least one shaped slot and a second holder coupled to the first holder, such that the first and second holder are sized and shaped to grasp the bone graft. A cutting guide is also included and has at least one slot. The cutting guide is coupled to one of the first holder and the second holder. The bone graft shaper also includes a bone saw sized and shaped to fit within at least one of the shaped slot and the cutting guide slot.

### TECHNICAL FIELD

The present invention relates generally to an instrument for use in orthopaedic surgery, and more particularly to an instrument for shaping bone graft.

### BACKGROUND

During the lifetime of a patient, it may be necessary to perform a total shoulder replacement procedure on the patient as a result of, for example, disease or trauma. In a total shoulder replacement procedure, a humeral component having a head portion is utilized to replace the natural head portion of the arm bone or humerus. The humeral component typically has an elongated intramedullary stem that is utilized to secure the humeral component to the patient's humerus. In such a total shoulder replacement procedure, the natural glenoid surface of the scapula is resurfaced or otherwise replaced with a glenoid component that provides a bearing surface for the head portion of the humeral component.

In response to shortcomings associated with two-piece glenoid component designs, a number of one-piece glenoid components have heretofore been designed. In regard to such one-piece designs, a body portion, having a bearing surface defined therein for receiving the head of the humeral component, has a number of attachment pegs integrally formed therewith. The attachment pegs are advanced and thereafter secured into a corresponding number of holes that are drilled in the glenoid surface of the scapula by use of bone cement. An example of such a one-piece glenoid component that is designed to be secured to the scapula by use of bone cement is disclosed in U.S. Pat. No. 5,032,132 issued to Matsen, III et al.

Certain one-piece glenoid components have been designed to include finned-pegs, such as described in U.S. Pat. No. 6,911,047, which is herein incorporated by reference in its entirety. The fins act as barbs when the pegs are inserted into holes drilled in the scapula, securing the glenoid component to the scapula.

Some one-piece glenoid components include steps or augments to fill in bone defects in the glenoid. Some stepped glenoid components are described in U.S. Pat. No. 6,699,289 entitled “Augmented Glenoid Component Having an Interrupted Surface and Associated Method for Securing the Augmented Glenoid Component to a Glenoid Surface of a Scapula”, filed on Dec. 31, 2001 and granted on Mar. 2, 2004, which is incorporated by reference herein in its entirety. The augment fits within a prepared defect of the glenoid. However, the stepped glenoids only come in certain sizes. Sometimes the defect is between sizes, or is larger than the largest size. In those instances, a surgeon may desire to use bone graft to fill the defect.

Currently, some surgeons utilize the resected humeral head to prepare the bone graft. However, there is not a shaper or other instrument that guides the surgeon in the preparation of the cuts. Instead, some surgeons make a trace of the defect on sterile paper and then shape the resected humeral head by hand. This takes a long time in the operating room and is very imprecise. Another concern is that, without the use of instruments, the surgeon often holds the resected head in his/her hand while cutting, risking injury to his/her hands.

Therefore, there is a need for an instrument to efficiently and adequately shape bone graft to fit a glenoid defect.

### SUMMARY OF THE INVENTION

According to one embodiment of the present invention, a bone graft shaper for shaping bone graft is provided. The bone graft shaper includes a first holder having at least one shaped slot and a second holder coupled to the first holder, such that the first and second holder are sized and shaped to grasp the bone graft. A cutting guide including at least one slot is also included. The cutting guide is coupled to one of the first holder and the second holder. A bone saw is sized and shaped to fit within at least one of the shaped slot and the cutting guide slot.

According to another embodiment of the present invention, a method for preparing bone graft is provided. The method includes resecting bone from a patient. The resected bone is clamped in a bone graft shaper, which includes a holder having at least one shaped slot and a cutting guide having at least one slot. A saw is used to cut through the at least one slot in the cutting guide and to cut through the at least one shaped slot of the holder.

According to yet another embodiment of the invention, a bone graft shaper for shaping a resected humeral head for use in a prepared glenoid is provided. The bone graft shaper includes a first holder having a plurality of curved slots and a second holder coupled to the first holder, such that the first and second holder are sized and shaped to grasp the resected humeral head. A cutting guide is also included and has a plurality of slots. The cutting guide is coupled to one of the first holder and the second holder. A bone saw is also included and is sized and shaped to fit within at least one of the plurality of curved slots and the plurality of cutting guide slots.

### BRIEF DESCRIPTION OF THE DRAWINGS

For a more complete understanding of the present invention and the advantages thereof, reference is now made to the following description taken in connection with the accompanying drawings.

FIG. 1 is a perspective view of a bone graft shaper according to one embodiment of the present invention.

FIG. 2 is a bottom view of the bone graft shaper of FIG. 1.

FIG. 3 is a flow chart illustrating the method of operating the bone graft shaper of FIG. 1.

FIG. 4 is a perspective view of a humeral head.

FIG. 5 is a perspective view of the humeral head of FIG. 4 after resection.

FIG. 6 is a top view of the bone graft shaper of FIG. 1 and the bisected, resected humeral head of FIG. 5 in use.

FIG. 7 is a bottom view of the bone graft shaper of FIG. 1 and the bisected, resected humeral head of FIG. 5 in use.

FIG. 8 is a side view of the bone graft shaper of FIG. 1 and the bisected, resected humeral head of FIG. 5 in use.

FIG. 9 is a perspective view of the bisected, resected humeral head after being shaped by the bone graft shaper.

FIG. 10 is a side view of a bone graft shaper according to another embodiment of the present invention.

FIG. 11 is a perspective view of the bone graft shaper of FIG. 10.

FIG. 12 is a side view of a holder of the bone graft shaper of FIG. 10.

### DETAILED DESCRIPTION OF THE INVENTION

Embodiments of the present invention and the advantages thereof are best understood by referring to the following descriptions and drawings, wherein like numerals are used for like and corresponding parts of the drawings.

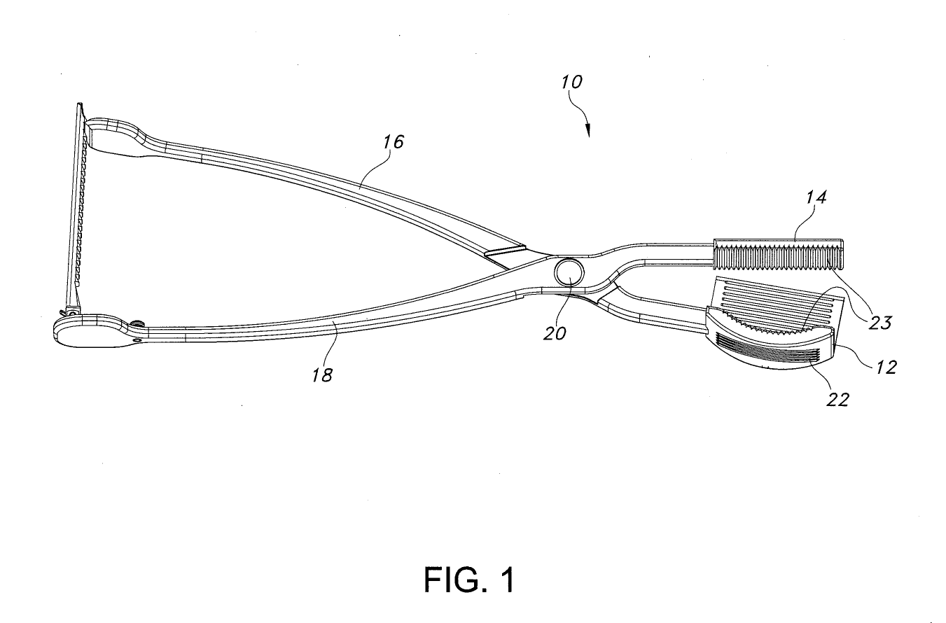

Referring now to FIG. 1, there is shown a bone graft shaper 10. The bone graft shaper 10 will be used to shape bone graft. Bone graft includes allograft, autograft or bone graft substitutes. The bone graft shaper 10 includes a first holder 12 and a second holder 14. The first and second holders 12, 14 are coupled to one another. In this case, the first and second holders 12, 14 include handles 16, 18 (respectively) and are pivotally connected via a pivot pin 20. The handles 16, 18 and the pivot pin 20 allow a user to open and close the first and second holders 12, 14 to grasp bone graft (as will be described in further detail below). As shown in FIG. 1, the first holder 12 includes at least one shaped slot 22. The shaped slot is dimensioned to fit the shape of the defect that the bone graft will fill. In this embodiment, the shaped slot 22 is curved. In other embodiments, it may be straight. In some embodiments, the curved-shaped slot 22 extends through the holder 12 perpendicular to the plane of the holder 12. In other embodiments, the shaped slot 22 may extend at angle that is more or less than 90° from the plane of the first holder 12. The angle allows for slanted cuts to better fill the defect. In the illustrated embodiment, each of the first and second holders 12, 14 include a plurality of teeth 23. Other embodiments may not include teeth 23. In yet other embodiments, only one of the first and second holders 12, 14 may include teeth. Other textures, such as knurling, grit blast, etc . . . may also be used.

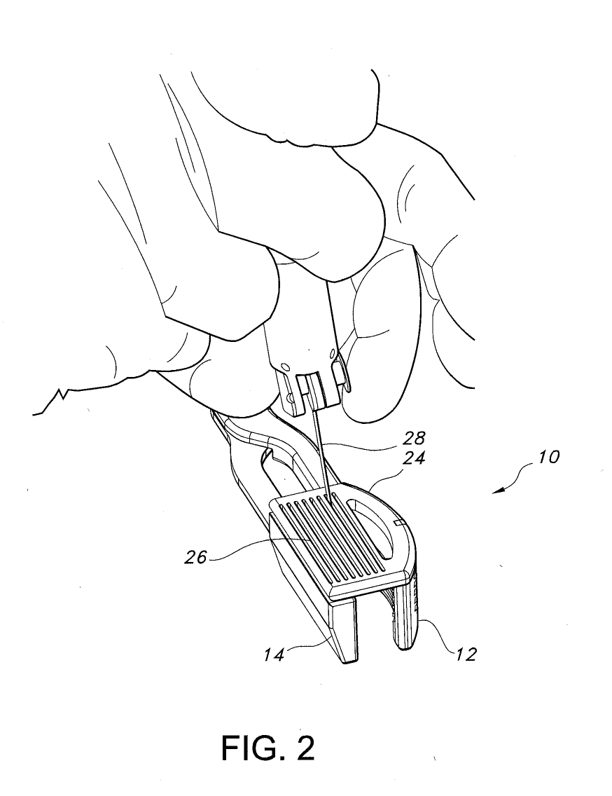

Turning now to FIG. 2, a bottom view of the bone graft shaper 10 is shown. The bone graft shaper 10 includes a cutting guide 24 that is coupled to the first holder 12. In other embodiments, the cutting guide 24 may be coupled to the second holder 14. As shown in FIG. 2, the cutting guide 24 is integral with the first holder 12 and extends perpendicularly from the first holder 12. In other words, the cutting guide 24 is of one piece with the first holder 12. In other embodiments, the cutting guide 24 may be integral with the second holder 14. In yet other embodiments, the cutting guide 24 may be a separate piece from the holder 12, 14 to which it is coupled.

As shown in FIG. 2, the cutting guide 24 includes at least one slot 26 for receiving a bone saw 28 during use. The bone saw 28 is also sized and shaped to fit within the shaped slot of the first holder 12 during use. In other embodiments, multiple bone saws 28 may be used, one to fit into the shaped slot 22 of the first holder 12 and one to fit in the slot 26 of the cutting guide 24.

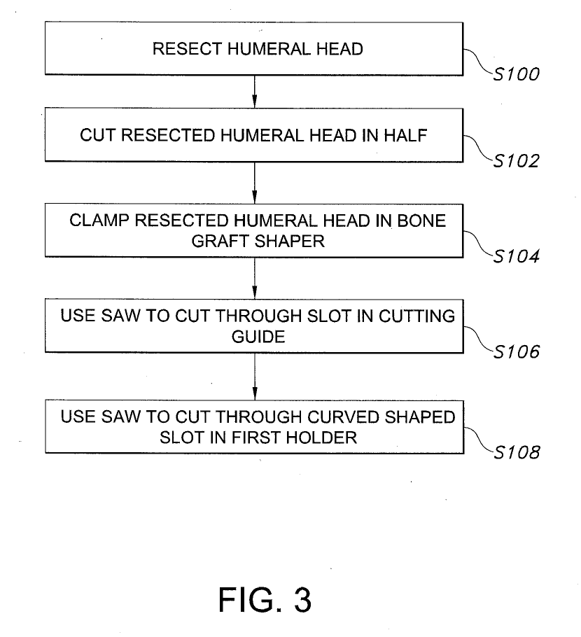

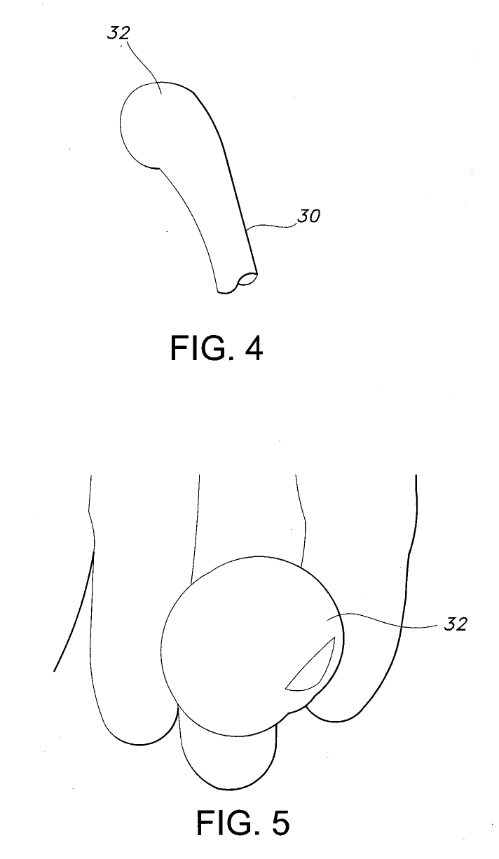

Turning now to FIGS. 3-9, a flow chart and illustrations demonstrating the method of using the bone graft shaper 10 is shown. First, as shown in FIG. 4, a humerus 30 is shown with a humeral head 32. Although the illustrated embodiment shows utilizing a resected humeral head, it should be understood that other types of bone graft may be used. As described above, the present invention can be used with allograft, autograft, or bone graft substitute. In some embodiments, cadaveric bone graft (allograft) may be used. Alternatively, autografts from other parts of the patient's body may also be used.

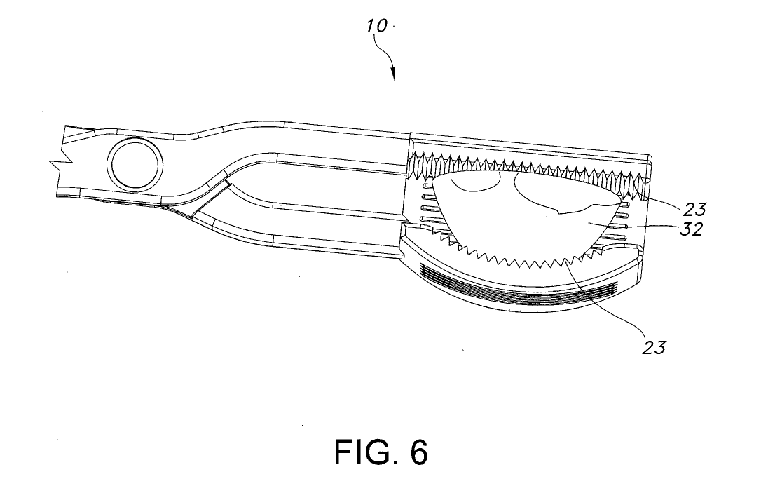

The humeral head 32 is resected as is known in the art (step s100). The resected humeral head 32 is shown in FIG. 5. The resected humeral head 32 is then cut in half at step s102. The bisected, resected humeral head 32 is then clamped in the bone graft shaper 10 as shown in FIG. 6 (step s104). As mentioned above, the handles 16, 18 of the first and second holders 12, 14 are pivotally connected allowing a user to open and close the first and second holders 12, 14 about the bisected, resected humeral head 32. Also, the first holder 12 is curved to more closely conform to the shape of the bisected, resected humeral head 32. In other embodiments, the shape of the first holder 12 may be different to conform to the type of bone being cut. The second holder 14 is flat to hug and/or fit the cut side of the bisected, resected humeral head 32. The second holder 14 could be a different shape depending upon the bone graft that is being shaped.

As shown in FIG. 6, the teeth 23 of the first and second holders 12, 14 grasp the bisected, resected humeral head 32, providing a secure hold. In some embodiments, there may not be teeth 23. In those embodiments, the friction between the first and second holders 12, 14 and the bisected, resected humeral head 32 may be enough to hold the resected humeral head in place.

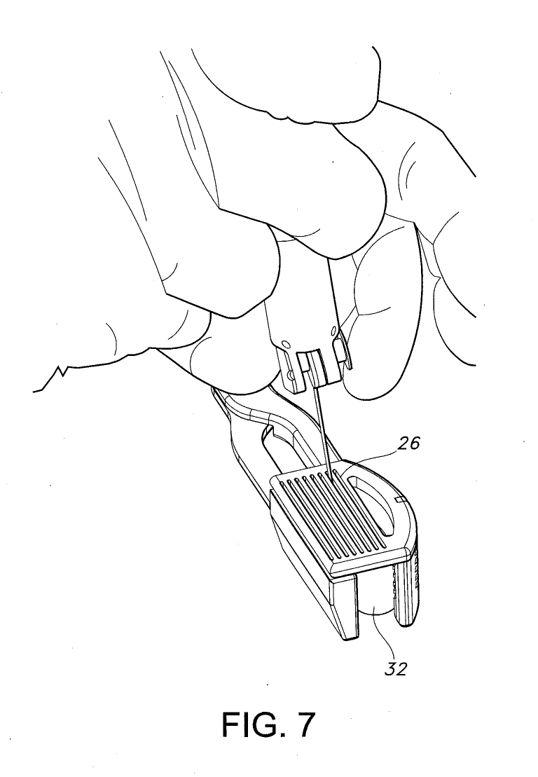

At step s106, the bone graft shaper is turned over and the cutting saw 28 is inserted through one of the plurality of slots 26. Each of the plurality of slots 26 corresponds to a different width. Because the bisected, resected head 32 is being shaped to fit a defect in the glenoid, the slots 26 define what the width of the resulting bone graft will be. This width will match the width of the prepared glenoid defect. In the embodiment illustrated in FIG. 7, there are a plurality of slots 26, each corresponding to a different width. In one embodiment, the slots 26 are spaced approximately 2 mm apart. In other embodiments, the slots 26 are spaced approximately 1 mm apart. In yet other embodiments, the slots 26 may not be evenly spaced or may be spaced apart differently (e.g., 3 mm, 4 mm, etc.). In some embodiments, there may be only a single slot 26. In other embodiments, there may be a different number of slots 26.

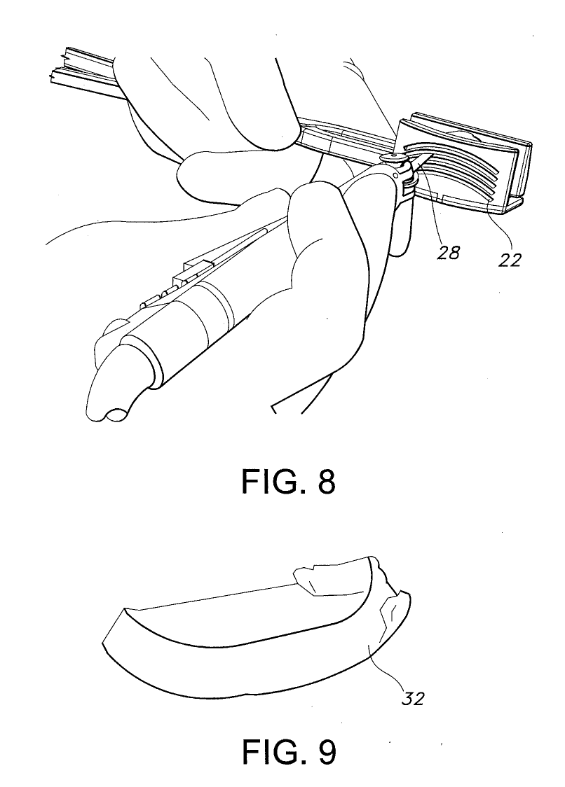

At step s108, the saw 28 is inserted through one of the plurality of shaped slots 22 as shown in FIG. 8. Each of the plurality of shaped slots 22 corresponds to a different height. Because the bisected, resected head 32 is being shaped to fit a defect in the glenoid, the slots define what the height or thickness of the resulting bone graft will be. This height or thickness will match correspond to the depth of the prepared glenoid defect. In the embodiment illustrated in FIG. 8, there are a plurality of slots 22, each corresponding to a different height. In one embodiment, the slots 22 are spaced approximately 2 mm apart. In other embodiments, the slots 22 are spaced approximately 1 mm apart. In yet other embodiments, the slots 22 may not be evenly spaced or may be spaced apart differently (e.g., 3 mm, 4 mm, etc.). In some embodiments, there may be only a single slot 22. In other embodiments, there may be a different number of slots 22.

The shaped bisected, resected head 32 is shown in FIG. 9. This shaped bisected, resected head (or bone graft) 32 will fit into the prepared glenoid defect and, after reaming and/or another form of bone prep such as drilling, will be able to receive a glenoid component (not shown).

Although the above steps state that the cutting guide slot 26 is cut first and that the shaped slot 22 is used second, the cutting may take place in either order.

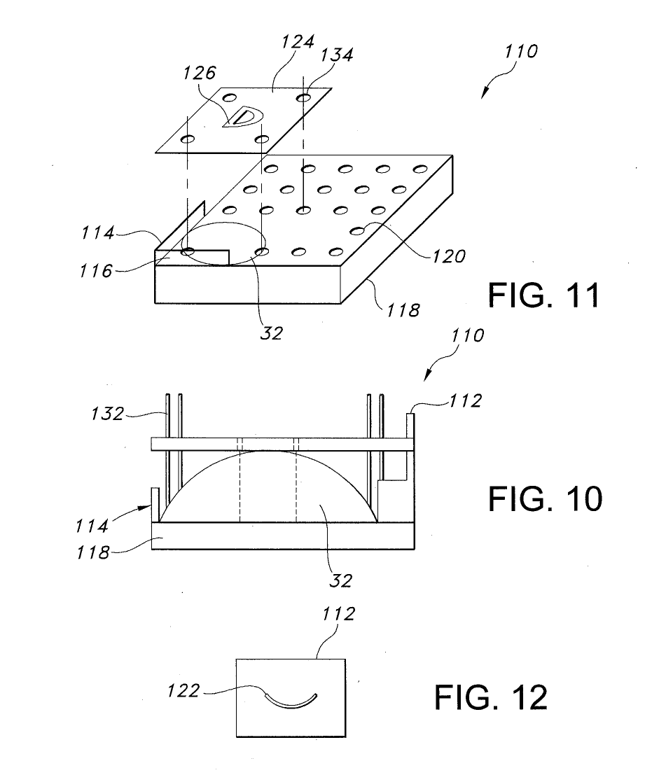

Turning now to FIGS. 10-12, another embodiment of a bone graft shaper 110 is illustrated. FIG. 10 is a side view of the bone graft shaper 110. The bone graft shaper 110 includes a first holder 112 and a second holder 114. In some embodiments, there may be a third holder 116 as well as a fourth holder (not shown). The shaper 110 also includes a base 118 that is coupled to the first, second, and third holders 112, 114, 116. The first, second, and third holders 112, 114, 116 all extend upwardly and are perpendicular to the base 118. The base 118 may also include a plurality of pin holes 120, which will be described in more detail below.

FIG. 12 is a side view of the first holder 112 and illustrates a shaped slot 122. In FIG. 11, a cutting guide 124 is shown. In this embodiment, the cutting guide 124 is a plate that is set on top of the first, second, and third holders 112, 114, 116. The cutting guide 124 is parallel with the base 118. The cutting guide 124 includes a slot 126 that may be used in conjunction with the saw 28 of FIG. 2. In this embodiment, the shaped slot 126 is D-shaped, although it may have other shapes depending on the final shape of the bone that is desired.

As with the bone graft shaper 10, the bone graft shaper 110 is used to shape a bone graft, which in this illustrated embodiment is a bisected, resected humeral head 32. As shown in FIG. 11, the bisected, resected humeral head 32 is placed on the base 118. The bisected, resected humeral head 32 is placed so as to abut the second and third holders 114, 116. The cutting guide 124 is slid on top of the second and third holders 114, 116 and locked into place via posts (or pins) 132 that slide through holes 134 in the cutting guide 124 and holes 120 in the base 118. The first holder 112 is then slid into place and, in some embodiments, locks into the cutting guide 124. A saw, such as the saw 28 from FIGS. 1-9, is inserted through the shaped slot 122 of the first holder 112 and the D-shaped slot 126 of the cutting guide 124 to shape the resected humeral head 32. In some embodiments, the resected humeral head 32 may be bisected prior to being placed in the cutting guide 124. The shaped slot 122 may extend perpendicular to the plane of the first holder 112. Alternatively, the shaped slot 122 may extend at an angle other than 90° to the plane of the first holder 112.

In some embodiments, the D-shaped slot 126 on the cutting guide 124 may not be d-shaped, but may be a one or a plurality of straight line slots, like those shown in the cutting guide 24 of FIGS. 1-9. In other embodiments, different shapes, such as circles, trapezoids, squares, rectangles, etc . . . may be used depending upon the type of cut needed. Also, in other embodiments, pins 132 may not be used to lock the holders 112, 114, 116 and the cutting guide 124 in place. Other known locking mechanisms such as snap locks, taper locks, etc . . . may be used.

The first holder 112 is shown with a single shaped slot 122. In other embodiments, there may be a plurality of shaped slots 122 as with the shaped slots 22 of FIGS. 1-9. In other embodiments, the first holder 112 may be adjustable within the base 118. In those embodiments, there may be a single shaped slot 122. However, because the first holder 112 is adjustable upwards and downwards relative to the base 118 (and thus the bisected, resected humeral head 32), the shaped slot 122 may be used to create different depths of the curved cut.

In some embodiments, the shapers 10, 110 may be made of stainless steel. In other embodiments, the shapers 10, 110 may be made of plastic. Other materials may also be used.

The above embodiments both describe using the bone graft applicator to shape bone graft using a resected humeral head. However, the bone graft shaper may also be used to shape bone graft using other resected bone, such as patellas, femoral heads, distal femurs, proximal tibias, etc. . . . It should also be reiterated that as stated above, that such a bone graft shaper would also work on artificial or cadaveric bone. The shaper of the present invention may also be used to create bone graft shaped to fit in other areas besides glenoids. For example, the bone graft could be shaped to fit within an acetabular cavity, a distal femur, or a proximal or distal tibia.

Although the present invention and its advantages have been described in detail, it should be understood that various changes, substitutions, and alterations could be made therein without departing from the spirit and scope of the present invention as defined by the appended claims.

We claim:

1. A bone graft shaper for shaping bone graft, the bone graft shaper comprising:
a first holder having at least one shaped slot;
a second holder coupled to the first holder, such that the first and second holder are sized and shaped to grasp the bone graft;
a cutting guide including at least one slot, the cutting guide coupled to one of the first holder and the second holder;
a bone saw sized and shaped to fit within at least one of the shaped slot and the cutting guide slot.

2. The bone graft shaper of claim 1, wherein the cutting guide is integrally connected to the one of the first holder and second holder.

3. The bone graft shaper of claim 2, wherein the cutting guide extends perpendicularly outward from the one of the first holder and the second holder.

4. The bone graft shaper of claim 1, wherein the cutting guide includes a plurality of slots.

5. The bone graft shaper of claim 1, wherein the first and second holders each include a handle sized and shaped to be grasped by a user and the handles are pivotally connected to one another.

6. The bone graft shaper of claim 1, wherein the bone graft is a bisected, resected humeral head and the first holder is curved to grasp the bisected, resected humeral head.

7. The bone graft shaper of claim 1, wherein at least one of the first holder and the second holder includes texture sized and shaped to grasp the bone graft.

8. The bone graft shaper of claim 7, wherein the texture is teeth.

9. The bone graft shaper of claim 1, wherein the shaped slot is angled in the first holder.

10. The bone graft shaper of claim 1, wherein the shaped slot is curved.

11. The bone graft shaper of claim 1, further including a base sized and shaped to receive the bone graft, wherein the first and second holders extend perpendicularly upward from the base.

12. The bone graft shaper of claim 10, wherein the cutting guide is a plate that is perpendicular to the first and second holders and parallel to the base.

13. The bone graft shaper of claim 11, wherein the slot in the plate is D-shaped to replicate the shape of a glenoid defect.

14. The bone graft shaper of claim 11, wherein the base and plate include holes and the bone graft shaper further includes posts that fit through the holes of the base and the plate, affixing the plate in a position relative to the base.

15. A method for preparing bone graft, the method comprising:
resecting bone from a patient;
clamping the resected bone in a bone graft shaper, the bone graft shaper including a holder having at least one shaped slot and a cutting guide having at least one slot;
using a saw to cut through the at least one slot in the cutting guide; and
using the saw to cut through the at least one shaped slot of the holder.

16. The method of claim 15, wherein the cutting guide includes a plurality of slots, and the method includes using the saw to cut through one of the plurality of slots, the one of the plurality of slots corresponding to a particular width of the resulting bone graft.

17. The method of claim 15, wherein the holder includes a plurality of shaped slots, and the method includes using the saw to cut through one of the plurality of slots, the one of the plurality of slots corresponding to a particular height of the resulting bone graft.

18. A bone graft shaper for shaping a resected humeral head for use in a prepared glenoid, the bone graft shaper comprising:
a first holder having a plurality of curved slots;
a second holder coupled to the first holder, such that the first and second holder are sized and shaped to grasp the resected humeral head;
a cutting guide including a plurality of slots, the cutting guide coupled to one of the first holder and the second holder;
a bone saw sized and shaped to fit within at least one of the plurality of curved slots and the plurality of cutting guide slots.

19. The bone graft shaper of claim 18, wherein the cutting guide is integrally connected to the one of the first holder and second holder.

20. The bone graft shaper of claim 18, further including a base sized and shaped to receive the resected humeral head, wherein he first and second holders extend perpendicularly upward from the base.
